(19) United States
(12) Patent Application Publication
(10) Pub. No.: US 2018/0327690 A1
(43) Pub. Date: Nov. 15, 2018

(54) BODY OIL CANDLE

(71) Applicant: Peter Harrison, Clear Lake, IA (US)

(72) Inventor: Peter Harrison, Clear Lake, IA (US)

(21) Appl. No.: 15/945,838

(22) Filed: Apr. 5, 2018

Related U.S. Application Data
(60) Provisional application No. 62/505,356, filed on May 12, 2017.

(51) Int. Cl. C11C 5/00; C11C 5/02; A61K 8/92; A61Q 13/00; A61Q 19/00; A61K 8/02

(52) U.S. Cl. CPC C11C 5/008; C11C 5/023; A61K 8/922; A61K 2800/87; A61Q 19/007; A61K 8/0216; A61Q 13/00

(57) ABSTRACT

A body oil/body butter candle having soy wax, coconut oil, body butter, and fragrance. After heating and blending each ingredient the composition/mixture is poured into a container having a wick and cooled until the composition has a partially solid soft butter consistency. By lighting the wick the composition is heated to a liquid skin safe state.

### CROSS REFERENCE TO RELATED APPLICATION

This application claims the benefit of U.S. Provisional Application No. 62/505,356 filed May 12, 2017.

### BACKGROUND OF THE INVENTION

This invention is directed to a candle comprising a body oil/body butter and more particularly to a candle comprised of a body oil/body butter in a semi-solid state.

Candles and body oil are well known in the art. While typically not combined, at least one attempt has been made to develop a candle for the delivery of body oil. In particular, U.S. Publ. No. 2005/0276826, incorporated herein by reference in its entirety, is directed to a delivery method and apparatus for body oil along with an associated body oil composition. While useful, the body oil composition is provided in a solid state at room temperature which is then heated to a liquid temperature that is skin safe and then applied to the skin and massaged into the skin. Accordingly, a need exists in the art for a body oil composition that addresses these deficiencies.

An objective of the present invention is to provide a body oil candle that at room temperature has a partially solid soft butter like consistency.

Another objective of the present invention is to provide a body oil candle that after being heated to a liquid state, upon cooling, returns to a partially solid butter like consistency.

These and other objectives will be apparent to those skilled in the art based upon the following written description, drawings and claims.

### SUMMARY OF THE INVENTION

A body oil candle having a composition or mixture that includes soy wax, coconut oil, body butter, and fragrance. After melting, blending, and cooling the ingredients, the composition has a lotion type, partially solid, soft butter like consistency.

By lighting a wick disposed within the composition, the composition is heated to a skin safe liquid state. Upon cooling, the composition returns to the soft butter like consistency.

### BRIEF DESCRIPTION OF THE DRAWINGS

FIG. 1 is a schematic view of an environment for a body oil candle; and

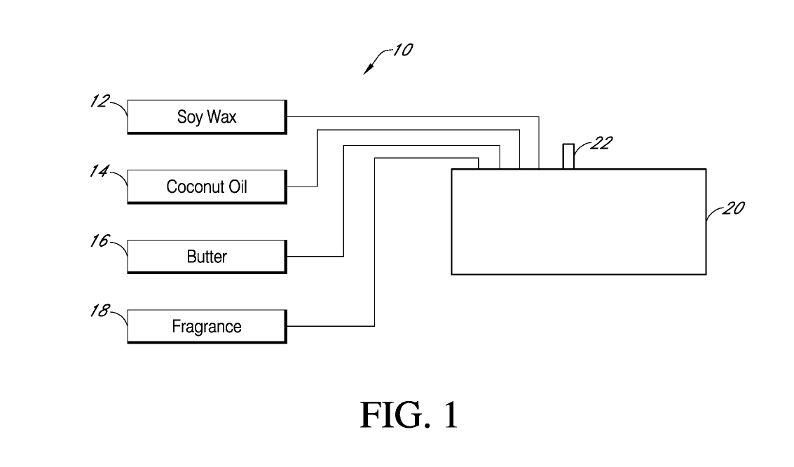

FIG. 2 is a flow diagram of a method of making a body oil candle.

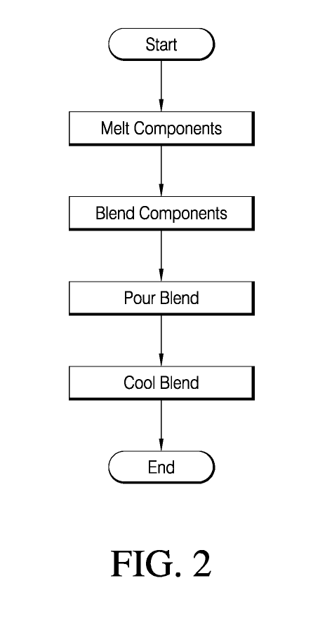

### DETAILED DESCRIPTION

Referring to the Figures, a body oil composition and candle 10 is made of a composition that includes soy wax 12, coconut oil 14, butter 16, and fragrance 18. In a preferred embodiment, the body oil composition 10 includes 50% soy wax 12, 16.5% coconut oil 14, 30% butter 16, and 3.5% fragrance 18.

To make the composition and candle 10, the soy wax 12, coconut oil 14, butter 16, and fragrance 18 are melted and then blended. Once blended, the composition 10 is poured into a container 20 having a wick 22. Once cooled, the composition 10 has a partially solid consistency that permits the composition to be scooped and applied to the skin. Alternatively, the wick 22 is lit which heats the composition to a liquid state at a skin safe temperature. The liquid composition 10 is then applied to and massaged into the skin. When the wick is blown out the composition cools and returns to the partially solid consistency.

What is claimed is:

1. A body oil/body butter candle, comprising:
a composition of soy wax, coconut oil, body butter, and fragrance having a consistency that permits the composition to be scooped and applied to skin.

2. The candle of claim 1 wherein the composition is disposed within a container and surround a wick.

3. The candle of claim 2 wherein the composition changes to a liquid state when the wick is lit.

4. The candle of claim 1 wherein the composition includes 50% soy wax, 16.5% coconut oil, 30% body butter, and 3.5% fragrance.

5. A body oil/body butter candle, consisting of:
a composition having soy wax, coconut oil, body butter, and fragrance.

6. The candle of claim 5 wherein the composition consist of 50% soy wax, 16.5% coconut oil, 30% body butter, and 3.5% fragrance.

7. A method of making and using a body oil/body butter candle comprising the steps of:
melting soy wax, coconut oil, body butter, and fragrance and then blending to form a mixture;
pouring the mixture into a container having a wick; and
cooling the mixture until the mixture has a consistency that permits the mixture to be scooped and applied to the skin.

8. The method of claim 7 further comprising the step of lighting the wick to heat the mixture to a liquid state at a skin safe temperature.

9. The method of claim 8 further comprising the step of applying the heated mixture to and massaging the liquid mixture into the skin.
